(19) United States
(12) Patent Application Publication
(10) Pub. No.: US 2025/0320444 A1
(43) Pub. Date: Oct. 16, 2025

(54) CELL CULTURE VESSEL AND CELL CULTURE METHOD

(71) Applicants: Koji TANABE, Palo Alto, CA (US); I Peace, Inc., Palo Alto, CA (US)

(72) Inventors: Koji TANABE, Palo Alto, CA (US); Ryoji HIRAIDE, Kyoto-shi (JP)

(73) Assignees: Koji TANABE, Palo Alto, CA (US); I Peace, Inc., Palo Alto, CA (US)

(21) Appl. No.: 18/552,776

(22) PCT Filed: Mar. 18, 2022
(86) PCT No.: PCT/JP2022/012699

§ 371 Date: Mar. 5, 2024

Related U.S. Application Data
(60) Provisional application No. 63/169,019, filed on Mar. 31, 2021.

(51) Int. Cl. C12M 1/00; C12M 1/04; C12M 1/24

(52) U.S. Cl. CPC C12M 23/40; C12M 23/08; C12M 23/24; C12M 23/38

(57) ABSTRACT

The cell culture vessel comprises a container and a container flow path connected to the container for delivering a fluid into the container, wherein at least a portion of the container is gas permeable.

### TECHNICAL FIELD

The present invention relates to cell technology and relates to a cell culture vessel and a cell culture method.

### BACKGROUND ART

Embryonic stem cells (ES cells) are stem cells established from an early human or mouse embryo. ES cells exhibit pluripotency and can differentiate into any of the cells present in an organism. At the present time, human ES cells can be used in cell transplantation therapies for numerous diseases, e.g., Parkinson's disease, juvenile diabetes, and leukemia. However, there are also obstacles to ES cell transplantation. In particular, ES cell transplantation can trigger immune rejection reactions like the rejection reactions that occur subsequent to unsuccessful organ transplants. In addition, there is a great deal of criticism and opposing opinion from an ethical standpoint to the utilization of ES cells established by the destruction of human embryos.

It was against this backdrop that Professor Shinya Yamanaka of Kyoto University succeeded in establishing induced pluripotent stem cells (iPS cells) through the introduction of 4 genes, i.e., OCT3/4, KLF4, c-MYC, and SOX2, into somatic cells. Professor Yamanaka was awarded the Nobel Prize in Physiology or Medicine in 2012 for this work (for example, refer to Patent Documents 1 and 2). iPS cells are ideal pluripotent cells being free of rejection reactions and ethical issues. iPS cells are thus expected to be used for cell transplantation therapies.

### CITATION LIST

### Patent Document

Patent Document 1: Japanese Patent No. 4183742
Patent Document 2: Patent Publication JP-A-2014-114997

### SUMMARY

### Technical Problem

There is desire for a method that can efficiently culture various types of cells, not just iPS cells. One object of the present invention is therefore to provide a cell culture vessel and a cell culture method that can efficiently culture cells.

### Solution to Problem

An aspect of the present invention provides a cell culture vessel that contains a container and a container flow path connected to the container for delivering a fluid into the container, wherein at least a portion of the container is gas permeable.

The gas permeable portion of the container in the aforementioned cell culture vessel may be impermeable to viruses.

The container in the aforementioned cell culture vessel may contain a housing having an opening and a lid for the opening.

The container flow path in the aforementioned cell culture vessel may be connected to the housing of the container.

At least a portion of the housing in the aforementioned cell culture vessel may be gas permeable.

The container flow path in the aforementioned cell culture vessel may be connected to the lid for the container.

At least a portion of the lid for the aforementioned cell culture vessel may be gas permeable.

The container flow path in the aforementioned cell culture vessel may be closable.

The aforementioned cell culture vessel may additionally contain a variable-volume container connected to the container flow path.

The aforementioned cell culture vessel may additionally contain a variable-volume container flow path that is connected to the variable-volume container.

The variable-volume container flow path in the aforementioned cell culture vessel may be closable.

The container flow path and the variable-volume container flow path in the aforementioned cell culture vessel may be aseptically connected.

The aforementioned cell culture vessel may be configured such that the volume of the variable-volume container changes when a fluid in the variable-volume container is transferred into the container.

The aforementioned cell culture vessel may be configured such that the volume of the variable-volume container changes when a fluid in the container is transferred into the variable-volume container.

Another aspect of the present invention provides a cell culture method comprising: preparing a cell culture vessel that contains a container and a container flow path connected to the container for delivering a fluid into the container, wherein at least a portion of the container is gas permeable; connecting, to the container flow path, a variable-volume container that contains a cell in its interior; transferring the cell into the container by contracting the variable-volume container; and culturing the cell in the container.

The aforementioned cell culture method may further include: closing the container flow path after the cell has been transferred into the container.

In the aforementioned cell culture method, a variable-volume container flow path may be connected to the variable-volume container and the variable-volume container may be connected to the container flow path by aseptically connecting the container flow path and the variable-volume container flow path.

The aforementioned cell culture method may further include: closing the variable-volume container flow path after the cell has been transferred into the container.

The aforementioned cell culture method may further include: transferring culture medium in the container into the variable-volume container by expanding the variable-volume container.

The container may be placed in the aforementioned cell culture method in a carbon dioxide incubator during the culture of the cell in the container.

Another aspect of the present invention provides a method for initializing a cell comprising: culturing a cell in the container of a cell culture vessel that contains a container and a container flow path connected to the container for delivering a fluid into the container, wherein at least a portion of the container is gas permeable; connecting, to the container flow path, a variable-volume container that contains an initialization factor in its interior; transferring the initialization factor into the container by contracting the variable-volume container; and initializing the cell in the container.

The aforementioned method for initializing a cell may further include: closing the container flow path after the initialization factor has been transferred into the container.

In the aforementioned method for initializing a cell, a variable-volume container flow path may be connected to the variable-volume container and the variable-volume container may be connected to the container flow path by aseptically connecting the container flow path and the variable-volume container flow path.

The aforementioned method for initializing a cell may further include: closing the variable-volume container flow path after the cell has been transferred into the container.

The container may be placed in the aforementioned method for initializing a cell in a carbon dioxide incubator for the initialization of the cell in the container.

Another aspect of the present invention provides a method for inducing cell differentiation, said method comprising: culturing a cell in the container of a cell culture vessel that contains a container and a container flow path connected to the container for delivering a fluid into the container, wherein at least a portion of the container is gas permeable; connecting, to the container flow path, a variable-volume container that contains a differentiation induction factor in its interior; transferring the differentiation induction factor into the container by contracting the variable-volume container; and inducing the differentiation of the cell in the container.

The aforementioned method for inducing cell differentiation may further include: closing the container flow path after the differentiation induction factor has been transferred into the container.

In the aforementioned method for inducing cell differentiation, a variable-volume container flow path may be connected to the variable-volume container and the variable-volume container may be connected to the container flow path by aseptically connecting the container flow path and the variable-volume container flow path.

The aforementioned method for inducing cell differentiation may further include: closing the variable-volume container flow path after the cell has been transferred into the container.

The container may be placed, in the aforementioned method for inducing cell differentiation, in a carbon dioxide incubator during induction of the differentiation of the cell in the container.

### Advantageous Effects of Invention

The present invention can thus provide a cell culture vessel and a cell culture method that can efficiently culture cells.

### BRIEF DESCRIPTION OF DRAWINGS

FIG. 1 is a schematic perspective diagram of a culture vessel according to an embodiment.

FIG. 2 is a schematic side view diagram of a culture vessel according to an embodiment.

FIG. 3a is a schematic perspective diagram and a schematic cross-sectional diagram of a culture vessel according to an embodiment.

FIG. 3b is a schematic perspective diagram and a schematic cross-sectional diagram of a culture vessel according to an embodiment.

FIG. 4 is a schematic side view diagram of a culture vessel according to an embodiment.

FIG. 5a is a schematic diagram of an aseptic connecting device according to an embodiment.

FIG. 5b is a schematic diagram of an aseptic connecting device according to an embodiment.

FIG. 5c is a schematic diagram of an aseptic connecting device according to an embodiment.

FIG. 6a is a schematic diagram of an aseptic connecting device according to an embodiment.

FIG. 6b is a schematic diagram of an aseptic connecting device according to an embodiment.

FIG. 7a is a schematic side view diagram of a culture vessel according to an embodiment.

FIG. 7b is a schematic side view diagram of a culture vessel according to an embodiment.

FIG. 7c is a schematic side view diagram of a culture vessel according to an embodiment.

FIG. 7d is a schematic side view diagram of a culture vessel according to an embodiment.

FIG. 8a is a schematic side view diagram of a culture vessel according to an embodiment.

FIG. 8b is a schematic side view diagram of a culture vessel according to an embodiment.

FIG. 9a is a schematic side view diagram of a culture vessel according to an embodiment.

FIG. 9b is a schematic side view diagram of a culture vessel according to an embodiment.

FIG. 9c is a schematic side view diagram of a culture vessel according to an embodiment.

FIG. 10a is a schematic side view diagram of a culture vessel according to an embodiment.

FIG. 10b is a schematic side view diagram of a culture vessel according to an embodiment.

FIG. 10c is a schematic side view diagram of a culture vessel according to an embodiment.

FIG. 10d is a schematic side view diagram of a culture vessel according to an embodiment.

FIG. 11a is a schematic side view diagram of a culture vessel according to an embodiment.

FIG. 11b is a schematic side view diagram of a culture vessel according to an embodiment.

FIG. 11c is a schematic side view diagram of a culture vessel according to an embodiment.

FIG. 11d is a schematic side view diagram of a culture vessel according to an embodiment.

FIG. 12a is a photograph that shows a cell according to an example.

FIG. 12b is a photograph that shows a cell according to an example.

FIG. 12c is a photograph that shows a cell according to an example.

FIG. 12d is a photograph that shows a cell according to an example.

### DESCRIPTION OF EMBODIMENTS

Embodiments of the present invention are described in the following. In the following description of the drawings, the same or analogous parts are represented by the same or analogous reference signs. However, the drawings are schematic. Therefore, the specific dimensions and so forth should be determined in light of the explanations that follow. In addition, parts having different dimensional relationships and/or ratios from each other are of course also present between the drawings.

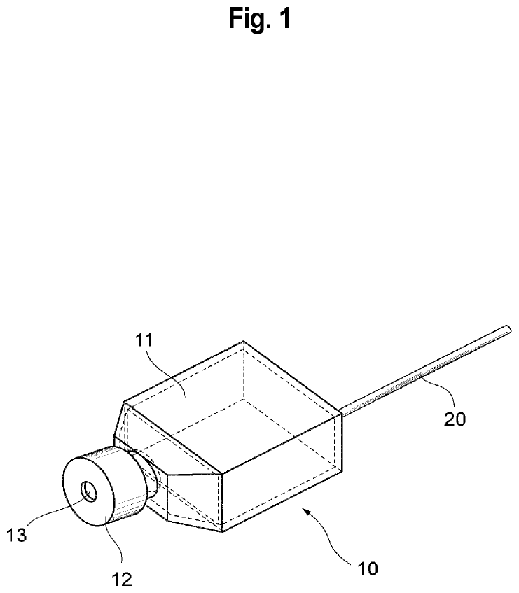

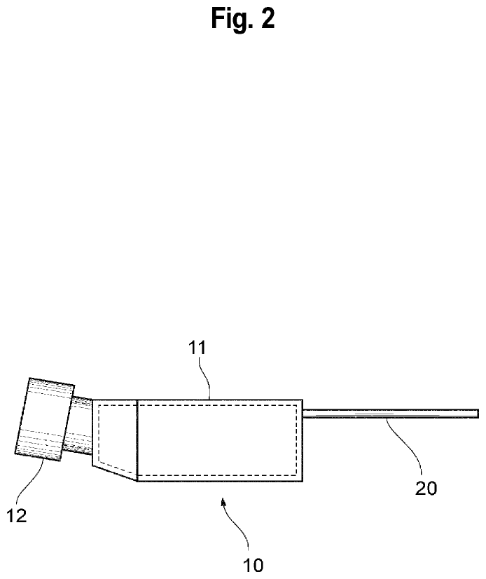

A cell culture vessel according to an embodiment, as shown in FIG. 1 and FIG. 2, comprises a container 10 and a container flow path 20 connected to the container 10 for delivering a fluid into the container 10. At least a portion of the container 10 is gas permeable. The gas permeable portion is permeable to gases and impermeable to liquids. The gas permeable portion is, for example, a filter.

The container 10, for example, comprises a housing 11 having an opening and with a lid 12 for the opening. At least a portion of the housing 11 may be gas permeable, and at least a portion of the lid 12 may be gas permeable. FIG. 1 shows an example in which a gas permeable filter 13 is disposed in the lid 12. The gas permeable portion, for example, is impermeable to dust, airborne particulates, bacteria, and viruses.

Cells are cultured within the housing 11. The size and shape of the housing 11 is not particularly limited as long as cells can be cultured in its interior. The container 10 may be a flask. Resins and glasses are examples of the material of the housing 11. The housing 11 may be transparent.

At least a portion of a surface constituting the housing 11 may be coated with a cell adhesion coating agent, or may not be coated. The cell adhesion coating agent can be exemplified by Matrigel, collagen, polylysine, fibronectin, vitronectin, gelatin, and laminin. Alternatively, in the case of suspension cell culture, at least a portion of a surface constituting the housing 11 may be coated with a coating agent that inhibits cell adhesion. Poly(2-hydroxyethyl methacrylate) is an example of a coating agent that inhibits cell adhesion. In addition, at least a portion of a surface constituting the housing 11 may be hydrophilic. The interior of the housing 11 may be subjected to a sterilization treatment. The sterilization treatment can be exemplified by high-pressure steam sterilization treatments, exposure to radiation, e.g., to gamma radiation, and sterilization treatments based on UV exposure.

The cells cultured in the culture vessel can be exemplified by somatic cells, but are not particularly limited. The cells can be exemplified by fibroblasts, nerve cells, retinal epithelial cells, hepatocytes, β-cells, renal cells, blood cells, dental pulp stem cells, keratinocytes, hair papilla cells, oral epithelial cells, megakaryocytes, T cells, NK cells, NKT cells, cartilage cells, myocardial cells, muscle cells, vascular cells, epithelial cells, factor-transduced cells, reprogrammed cells, and stem cells, although there is no particular limitation to these. The stem cells can be exemplified by mesenchymal stem cells, somatic stem progenitor cells, pluripotent stem cells, ES cells, and iPS cells, although there is no particular limitation to these.

The culture medium for cell culture is selected as appropriate in correspondence to the cell type. For example, when the cells are somatic cells, a somatic cell culture medium, e.g., a differentiated cell culture medium, is selected. When the cells are stem cells, a stem cell culture medium adapted to stem cells is selected. The culture medium may be a gel, a liquid, or a flowable solid. Flowable solids can be exemplified by agar and temperature-sensitive gels.

When the culture medium is a gel, the culture medium may contain a polymer compound. This polymer compound may be, for example, at least one selection from the group consisting of gellan gum, deacylated gellan gum, hyaluronic acid, rhamsan gum, diutan gum, xanthan gum, carrageenan, fucoidan, pectin, pectic acid, pectinic acid, heparan sulfate, heparin, heparitin sulfate, keratosulfate, chondroitin sulfate, dermatan sulfate, rhamnan sulfate, and salts of the preceding. The culture medium may also contain methyl cellulose.

Alternatively, the culture medium may contain a small amount of a temperature-sensitive gel selected from poly(glycerol monomethacrylate) (PGMA); poly(2-hydroxypropyl methacrylate) (PHPMA); poly(N-isopropylacrylamide) (PNIPAM); and amine-terminated, carboxylic acid-terminated, maleimide-terminated, N-hydroxysuccinimide (NHS) ester-terminated, or triethoxysilane-terminated poly(N-isopropylacrylamide-co-acrylamide), poly(N-isopropylacrylamide-co-acrylic acid), poly(N-isopropylacrylamide-co-butyl acrylate), poly(N-isopropylacrylamide-co-methacrylic acid), poly(N-isopropylacrylamide-co-methacrylic acid-co-octadecyl acrylate), and N-isopropylacrylamide. In the present disclosure, gel-form culture medium or gel culture medium encompasses polymer culture media.

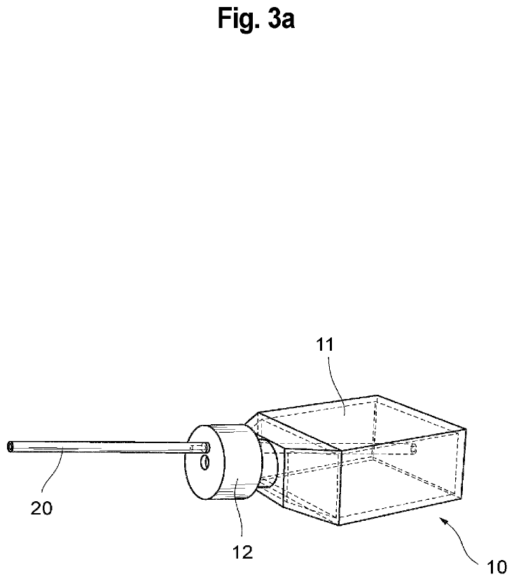

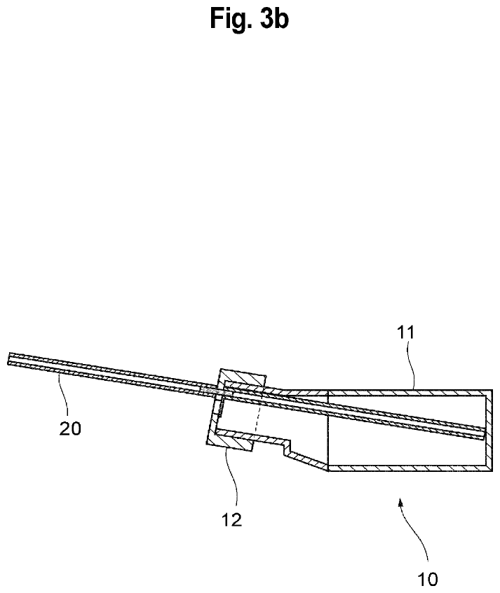

The container flow path 20 may be connected to the housing 11 of the container 10, and, as shown in FIG. 3, it may be connected to the lid 12 of the container 10. There are no particular limitations on the region where the container flow path 20 for the housing 11 of the container 10 is connected. For example, the container flow path 20 may be connected at a corner, or near a corner, of the housing 11 of the container 10, and may be connected above a cell adhesion surface of the housing 11 of the container 10. The container flow path 20 may be inserted into the interior of the housing 11 of the container 10. The container flow path 20 may reach to the bottom surface of the interior of the housing 11 of the container 10. This can facilitate removal of liquid near the bottom surface. The container flow path 20 may reach to an angle, or the neighborhood of an angle, of the bottom surface of the interior of the housing 11 of the container 10. There are also no particular limitations on the region where the container flow path 20 for the lid 12 of the container 10 is connected.

The container flow path 20 is, for example, a flexible tube. The container flow path 20 is composed of, for example, a resin. The resin may be, for example, a synthetic resin. This synthetic resin can be exemplified by polyvinyl chloride. The container flow path 20 is composed, for example, of a heat-sealable material. The container flow path 20 is, for example, closable.

For example, when the container flow path 20 is composed of a resin, the container flow path 20 is closed by pinching the container flow path 20 with a pressure-application device while heating. For example, by producing the housing 11 in a clean environment, connecting the housing 11 and the container flow path 20 in a clean environment, and closing the container flow path 20, a clean environment can be maintained in the interior of the container flow path 20 and the interior of the housing 11. The method for closing the container flow path 20 is not limited to the preceding, and, for example, the following may be used: light processing, laser light processing, friction processing, rubbing processing, processing with heat not accompanied by the application of pressure, and processing with the application of pressure not accompanied by heating. For example, the container flow path 20 may be pinched with, e.g., a clip.

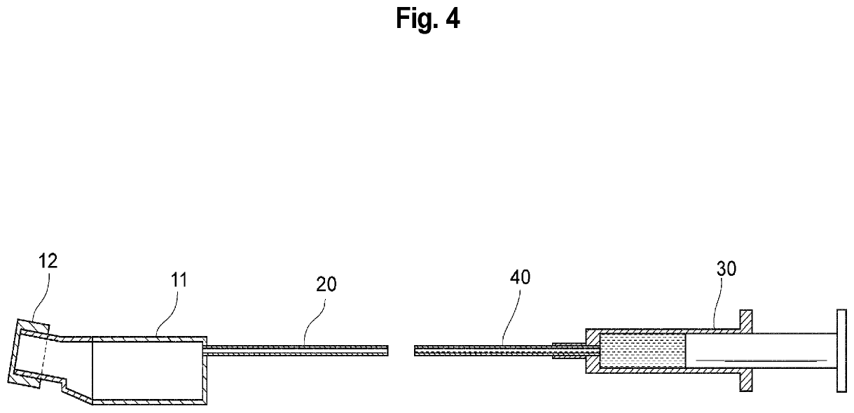

As shown in FIG. 4, the cell culture vessel according to the embodiment may further comprise a variable-volume container 30 connected to the container flow path 20. A variable-volume container flow path 40 may be connected to the variable-volume container 30, and the variable-volume container 30 may be connected to the container flow path 20 through the variable-volume container flow path 40.

The variable-volume container 30 contains, for example, a cell-containing solution. The variable-volume container 30 containing a cell-containing solution may be placed, until connection to the container flow path 20, in a temperature-controlled chamber that can be set to a temperature suitable for the cells.

The variable-volume container 30, for example, may comprise a syringe that holds a fluid and with a plunger that is inserted in the syringe and can move within the syringe, and the volume within the syringe that can hold a fluid may be changeable due to the movement of the plunger. Alternatively, the variable-volume container 30 may be a flexible bellows or bag.

The variable-volume container flow path 40 is, for example, a flexible tube. The variable-volume container flow path 40, for example, is composed of a resin. The resin is, for example, a synthetic resin. The synthetic resin can be exemplified by polyvinyl chloride. The variable-volume container flow path 40, for example, is closable.

For example, a container flow path 20 with a closed end may be connected by an aseptic connecting device with a variable-volume container flow path 40 with a closed end, to give an open passage from the container flow path 20 to the variable-volume container flow path 40.

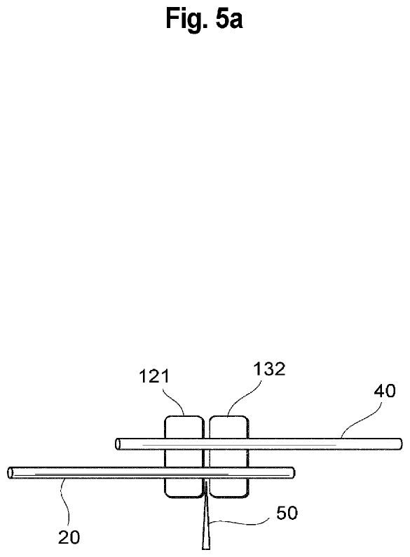

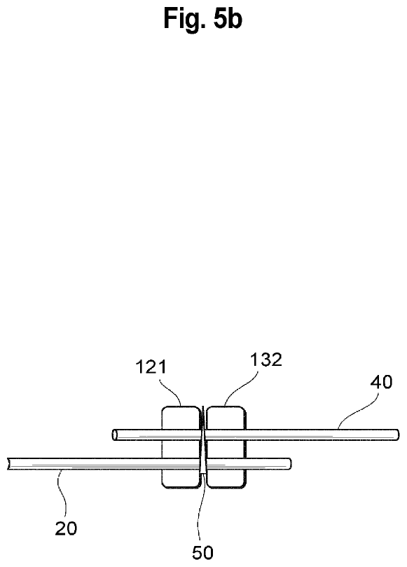

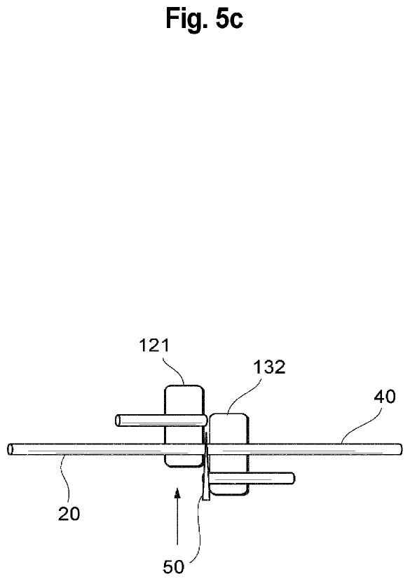

As shown in FIG. 5(a), the aseptic connecting device comprises, for example, holders 121, 132, which hold the container flow path 20 and the variable-volume container flow path 40 aligned in parallel over a segment, and with a movable cutter 50 between the holders 121, 132. The cutter 50 can be heated. As shown in FIG. 5(b), the cutter 50 moves between the holders 121, 132 and melts and cuts both the container flow path 20 and the variable-volume container flow path 40. The melted and cut portion of the container flow path 20 and the melted and cut portion of the variable-volume container flow path 40 are each closely adhered to a side of the cutter 50 and outside air does not infiltrate into the interior of the container flow path 20 or the variable-volume container flow path 40.

As shown in FIG. 5(c), at least one of the holders 121, 132 is displaced while the melted and cut portions of each of the container flow path 20 and the variable-volume container flow path 40 remain closely adhered to a side of the cutter 50, and the melted and cut portions of the container flow path 20 and the variable-volume container flow path 40 are thus arranged along a single line.

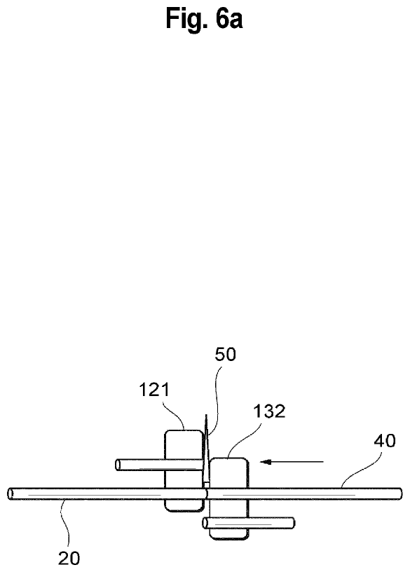

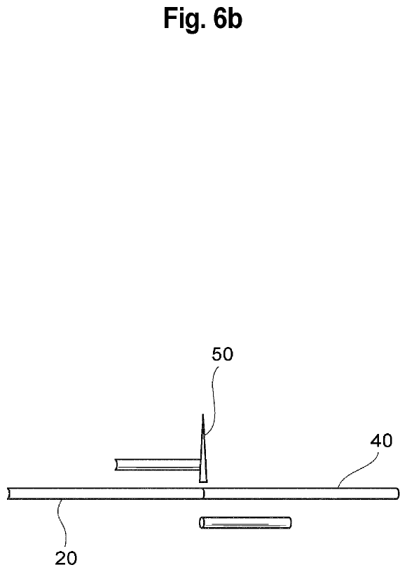

As shown in FIG. 6(a) and FIG. 6(b), the melted and cut portion of the container flow path 20 is joined with the melted and cut portion of the variable-volume container flow path 40 at the same time that the cutter 50 residing between the respective melted and cut portions of the container flow path 20 and variable-volume container flow path 40 is removed. As a consequence, the container flow path 20 can be aseptically connected or joined to the variable-volume container flow path 40 without causing the infiltration of outside air into the interior of the container flow path 20 or the variable-volume container flow path 40. The container flow path 20 and the variable-volume container flow path 40 are subsequently removed from the holders 121, 132.

The method for connecting the container flow path 20 with the variable-volume container flow path 40 is, however, not particularly limited, and, for example, the container flow path 20 may be connected with the variable-volume container flow path 40 using an aseptic connector.

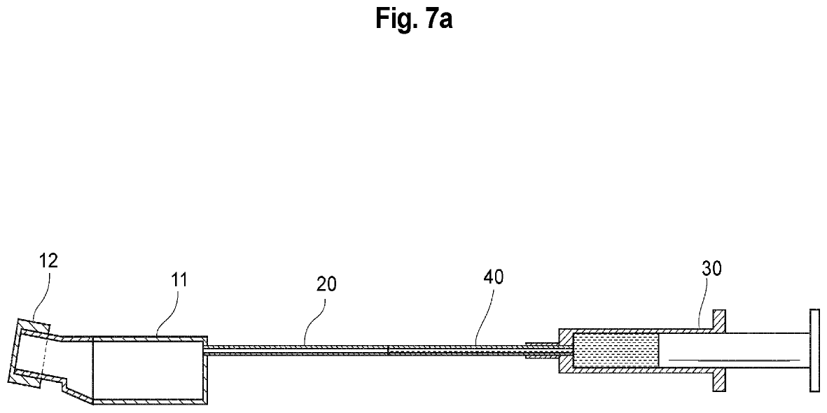

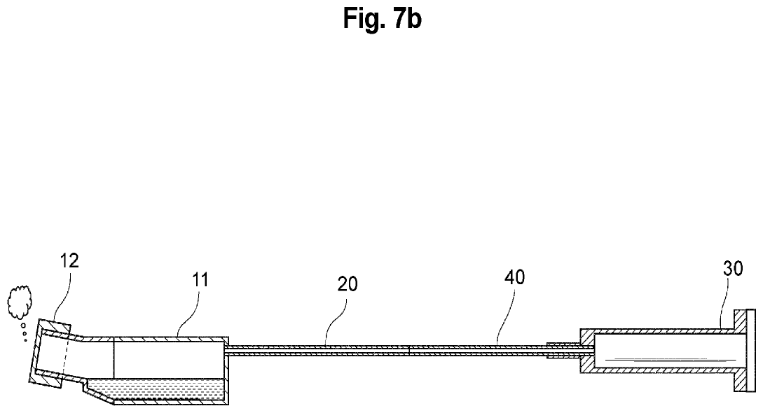

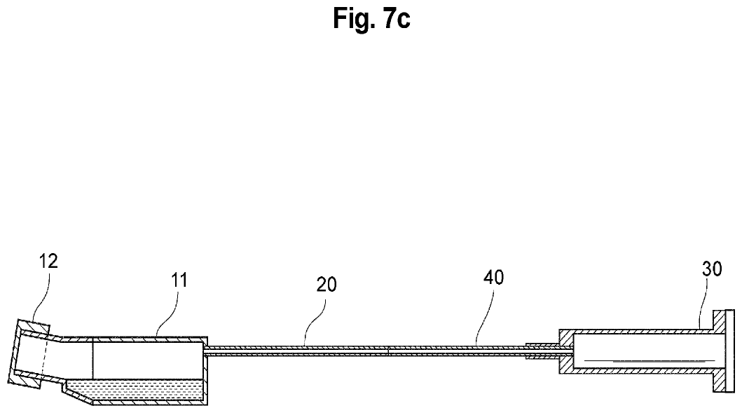

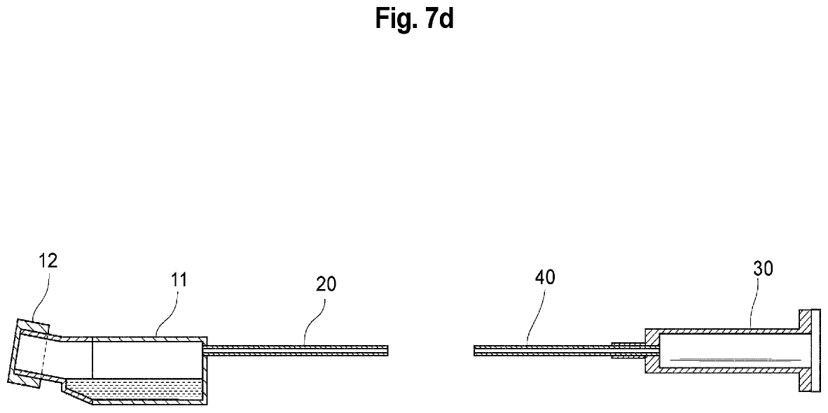

When the volume of the variable-volume container 30 containing in its interior a cell-containing solution, shown in FIG. 7(a), is changed by contraction, the cell-containing solution in the variable-volume container 30 is, as shown in FIG. 7(b), transferred into the housing 11 through the variable-volume container flow path 40 and the container flow path 20. In addition, the gas, e.g., air, in the housing 11 is discharged through the gas permeable portion. The cell-containing solution may be filled into the housing 11 so that an air layer does not remain in the housing 11. However, fine bubbles may remain in the solution filled into the housing 11 as long as an air layer does not remain. The cell-containing solution may be filled into the housing 11 such that an air layer remains present within the housing 11.

After the cell-containing solution has been transferred into the housing 11, the cells may be cultured with the variable-volume container 30 continuing to be connected to the container flow path 20. Alternatively, as shown in FIG. 7(c), for example, cell culture may be carried out with at least one of the container flow path 20 and the variable-volume container flow path 40 closed. Thermocompression is an example of a method for closing at least one of the container flow path 20 and the variable-volume container flow path 40. As shown in FIG. 7(d), the variable-volume container 30 may be removed from the housing 11 after the at least one of the container flow path 20 and the variable-volume container flow path 40 has been closed. After the removal of the variable-volume container 30, the cell culture vessel may be placed in an incubator that can be set to a temperature and carbon dioxide concentration suitable for cell culture. Carbon dioxide may be introduced into the container 10 through the gas permeable portion of the container 10. In addition, the cell culture vessel may be placed in a low-oxygen environment in order to bring the interior of the container 10 into a low oxygen state.

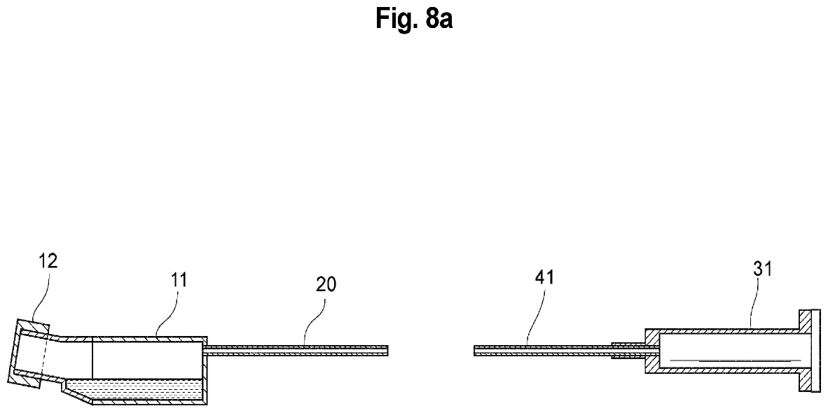

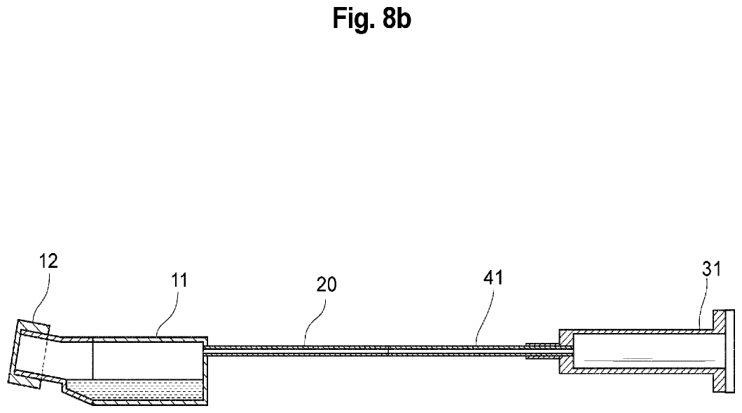

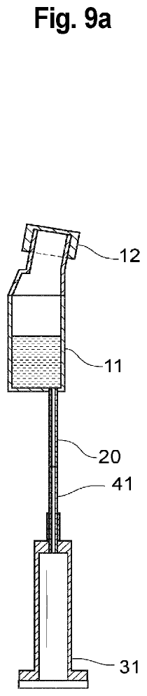

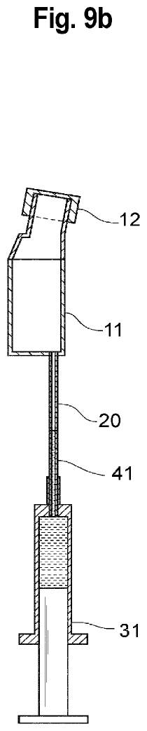

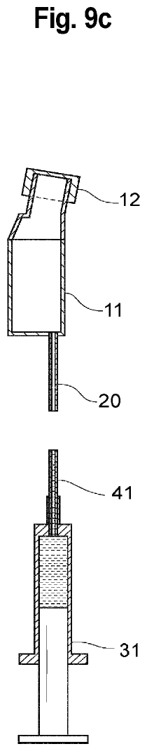

For example, when the culture medium in the housing 11 is to be exchanged during the culture within the housing 11 of cells that are undergoing adherent culture, as shown in FIG. 8(a) and FIG. 8(b), an expandable variable-volume container 31 is connected to the container flow path 20 through a variable-volume container flow path 41. As shown in FIG. 9(a) and FIG. 9(b), when the volume of the variable-volume container 31 is changed by expansion, the culture medium in the housing 11 is transferred into the variable-volume container 31 through the container flow path 20 and the variable-volume container flow path 41. At this time, the cell-adhered surface of the housing 11 may brought into parallel with the direction of gravity. Then, as shown in FIG. 9(c), the variable-volume container 31 is removed from the housing 11. This is followed by the introduction of fresh culture medium into the housing 11 using the same method as the method described in FIG. 4 to FIG. 7. The culture medium-containing variable-volume container 30 may be placed, until connection to the container flow path 20, in a temperature-controlled chamber that can be set to a temperature suitable for the culture medium. A temperature suitable for the culture medium may be 4° C.

Culture medium exchange may be carried out a plurality of times. In addition, depending on the status of the cells, culture medium exchange may be carried out while changing the type of culture medium.

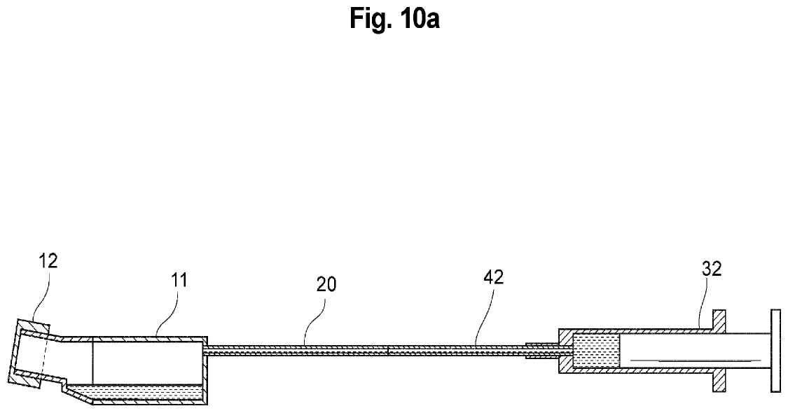

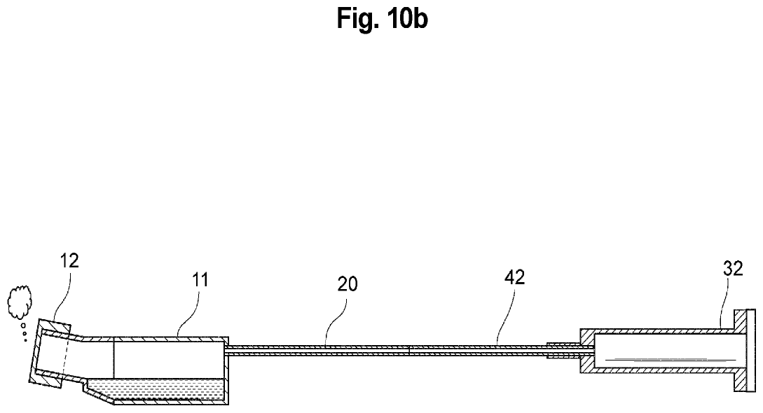

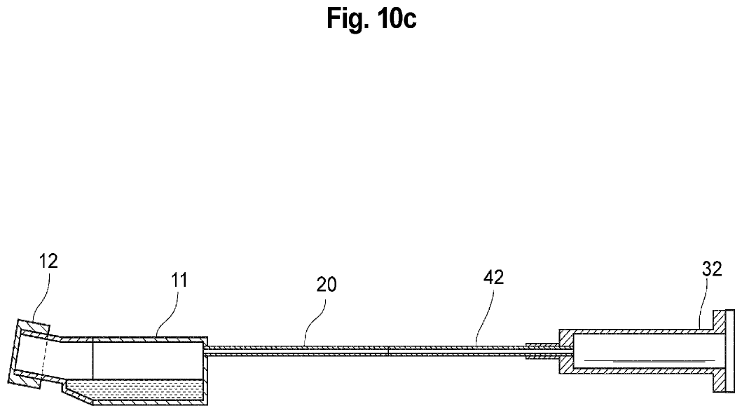

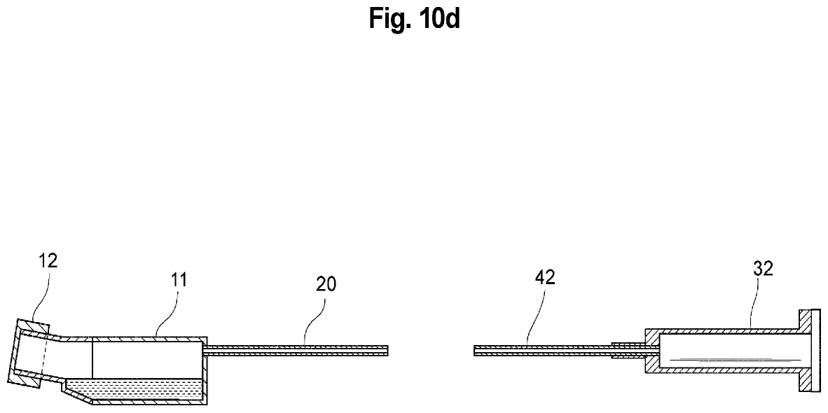

When factors are introduced to the cells cultured in the housing 11, as shown in FIG. 10(a) a variable-volume container 32 containing in its interior a factor-containing solution is connected through a variable-volume container flow path 42 to the container flow path 20. The variable-volume container 32 containing the factor-containing solution may be placed, until connection to the housing 11, in a temperature-controlled chamber that can be set to a temperature suitable for the factor.

The factor may be a nucleic acid, e.g., DNA, RNA, oligonucleotides, and so forth, or may be a protein, or may be a compound, or may be a virus. The DNA may be plasmid DNA. The RNA may be mRNA, siRNA, or miRNA. The RNA may be a modified RNA or may be an unmodified RNA. The nucleic acid may be incorporated in a vector. The vector can be exemplified by plasmids, retroviruses, lentiviruses, adenoviruses, adeno-associated viruses, episomal vectors, and Sendai viruses. The protein may be a nuclease protein, e.g., Cas9 protein. The virus may be a lentivirus. The factor may be an induction factor that induces a cell in a first state into a cell in a second state. The factor may be a hormone, growth factor, or low-molecular-weight compound.

In the present disclosure, induction refers to, for example, reprogramming, initialization, transformation, transdifferentiation or lineage reprogramming, differentiation induction, and cell fate reprogramming. Factors that induce cells other than pluripotent stem cells to be pluripotent stem cells are referred to as reprogramming factors. Reprogramming factors include, for example, OCT3/4, SOX2, KLF4, and c-MYC. Reprogramming factors, for example, also include compounds and growth factors, e.g., bFGF, TGF-β, and so forth. Factors that induce certain cells to be differentiated cells are referred to as differentiation induction factors. Differentiation induction factors induce stem cells to be differentiated cells. Alternatively, differentiation induction factors induce non-stem cell somatic cells to be different somatic cells. Differentiation induction factors include growth factors, e.g., activin, bone morphogenic proteins, FGF, and so forth, and compounds such as GSK inhibitors and smad inhibitors. In direct reprogramming, a non-stem cell somatic cell is induced to be a different non-stem cell somatic cell.

Examples of factors that induce cells to be nervous system cells are the ASCL family, DLX family, MYT family, NeuroD family, SOX family, and NGN family. The ASCL family can be exemplified by ASCL1. The DLX family can be exemplified by DLX2. The MYT family can be exemplified by MYT1L. The NGN family can be exemplified by NGN2. The nervous system cells can be exemplified by neurons, neural stem cells, and neural progenitor cells. The neurons can be exemplified by inhibitory neurons, excitatory neurons, dopamine-producing neurons, cranial nerves, mediated nerves, and optic nerves. Alternatively, the nervous system cells may be motor neurons, oligodendrocyte progenitor cells, astrocytes, oligodendrocytes, and so forth.

Factors that induce cells to be cardiomyocytes can be exemplified by the GATA family, MEF family, TBX family, MYOCD family, MESP family, and miR-133 family. The GATA family can be exemplified by GATA4A. The MEF family can be exemplified by MEF2C. The TBX family can be exemplified by TBX5. The MESP family can be exemplified by MESP1.

As shown in FIG. 10(b), upon changing, by contraction, the volume of the variable-volume container 32 containing in its interior the factor-containing solution, the factor-containing solution in the variable-volume container 32 is transferred into the housing 11 through the variable-volume container flow path 42 and the container flow path 20. As a result, the factors are brought into contact with the cells in the housing 11 and the factors are introduced into the cells.

After the factor-containing solution has been transferred into the housing 11, the factors may be introduced into the cells while keeping the variable-volume container 32 connected to the container flow path 20. Alternatively, for example, as shown in FIG. 10(c) and FIG. 10(d), the container flow path 20 or the variable-volume container flow path 42 may be closed and the variable-volume container 32 may then be removed from the housing 11. After removal of the variable-volume container 32, the housing 11 may be placed in an incubator that can be set to a temperature and carbon dioxide concentration suitable for the introduction of the factor.

Introduction of the factors into the cells may be carried out a plurality of times. In addition, after the introduction of the factors to the cells, the factor-containing solution in the housing 11 may be exchanged for culture medium using the same procedure as for culture medium exchange, and cell culture may be continued. Culture medium exchange may also be repeated using the procedure described above. This culture includes initialization culture and expansion culture.

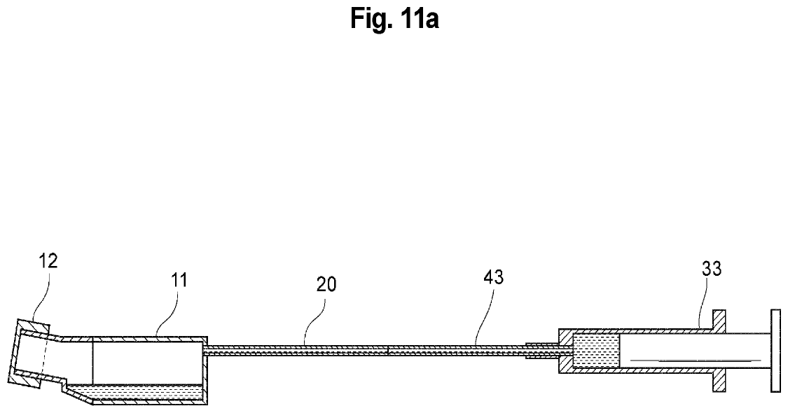

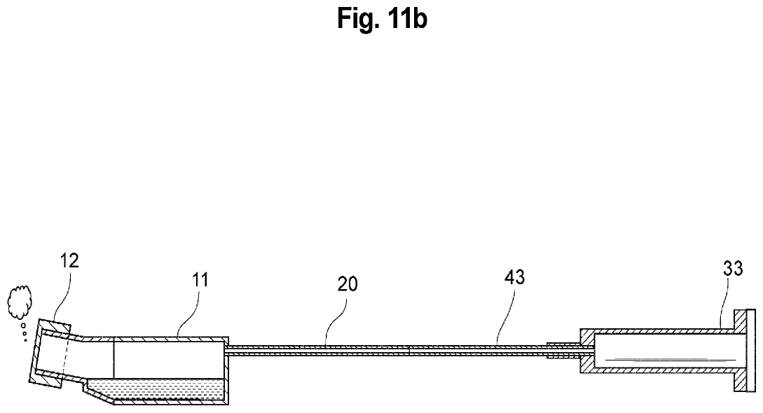

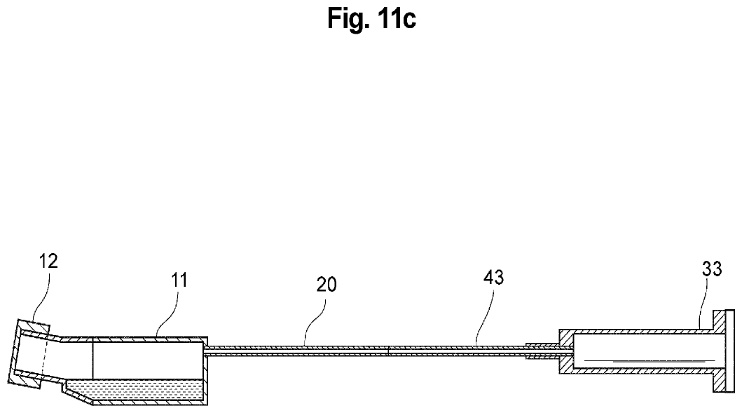

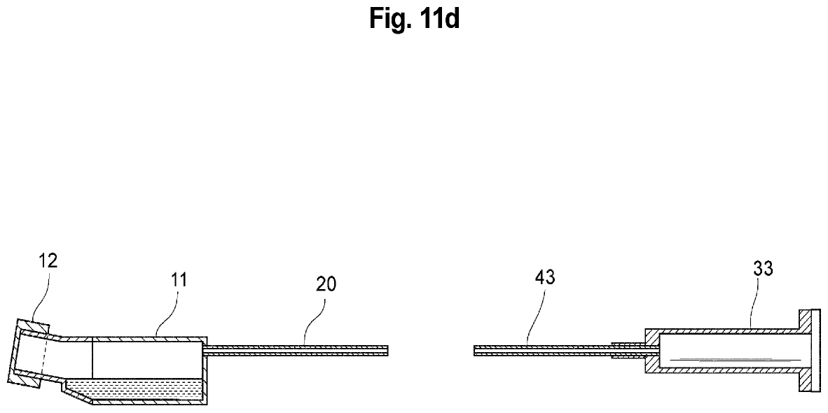

When the cells cultured in the housing 11 are detached from the housing 11, as shown in FIG. 11(a), a variable-volume container 33 containing a detachment agent-containing solution in its interior is connected to the container flow path 20 through a variable-volume container flow path 43. The detachment agent can be exemplified by trypsin, TrypLE Select, acutase, and EDTA. The variable-volume container 33 containing the detachment agent-containing solution may be placed, until connection to the housing 11, in a temperature-controlled chamber that can be set to a temperature suitable for the detachment agent.

As shown in FIG. 11(b), when the volume of the variable-volume container 33 containing the detachment agent-containing solution in its interior is changed by contraction, the detachment agent-containing solution in the variable-volume container 33 is transferred into the housing 11 through the variable-volume container flow path 43 and the container flow path 20. The detachment-containing solution is thereby brought into contact with the cells in the housing 11.

After the detachment agent has been transferred into the housing 11, the detachment agent may be contacted with the cells while the variable-volume container 33 remains connected to the housing 11. As shown in FIG. 11(c) and FIG. 11(d), the variable-volume container 33 may be removed from the housing 11 after the container flow path 20 or the variable-volume container flow path 43 has been closed. After the variable-volume container 33 has been removed, the housing 11 may be placed in an incubator that can be set to a temperature and carbon dioxide concentration suitable for cell detachment. After this, the detachment agent in the housing may be suctioned off by the same method as for the culture medium removal method described using FIG. 8 and FIG. 9. That is, when an expandable variable-volume container is connected through a variable-volume container flow path to the container flow path and the volume of the variable-volume container is expanded, the detachment agent in the housing is transferred into the variable-volume container through the container flow path and the variable-volume container flow path.

After the detachment agent has been removed, a culture solution may be introduced into the housing 11 and the cells may be detached from the housing 11 by shaking the housing 11. The detached cells may be seeded into the same or a different housing 11 to subculture the cells. In this subculturing, the recovered cells may be seeded without colony picking into a housing without classification. For recovery of the cells in a housing 11, the lid 12 may be opened and the cells may be recovered through the opening in the housing 11. Alternatively, the cell suspension within the housing may be suctioned off using the same method as the method for culture medium removal described using FIG. 8 and FIG. 9. That is, when an expandable variable-volume container is connected through a variable-volume container flow path to the container flow path and the volume of the variable-volume container is expanded, the cell suspension in the housing is transferred into the variable-volume container through the container flow path and the variable-volume container flow path.

For example, when a coating is executed within the housing 11, a variable-volume container containing the coating agent in its interior is connected to the container flow path 20 through the variable-volume container flow path 41. When the volume of the variable-volume container containing the coating agent in its interior is changed by contraction, the coating agent in the variable-volume container 32 is transferred into the housing 11 through the variable-volume container flow path and the container flow path. The interior of the housing 11 is thereby coated. The coating agent, for example, is introduced for at least 1 hour at room temperature into the housing 11. Alternatively, the coating agent, for example, is introduced for at least 6 hours at 4° C. into the housing 11. Then, in order to recover the excess coating agent in the housing 11, the lid 12 may be opened and the coating agent may be recovered through the opening in the housing 11. Alternatively, the coating agent within the housing may be suctioned off using the same method as the method for culture medium removal described using FIG. 8 and FIG. 9. That is, when an expandable variable-volume container is connected through a variable-volume container flow path to the container flow path and the volume of the variable-volume container is expanded, the coating agent in the housing is transferred into the variable-volume container through the container flow path and the variable-volume container flow path.

### Example

A flask housing with an interior coated with laminin 511 was prepared. A lid was placed on the opening in the housing, and a resin tube functioning as a container flow path was connected to the housing. A filter for gas permeation was also disposed in the lid. Human peripheral blood mononuclear cells were suspended in a blood cell culture medium; the number of mononuclear cells was measured using a hemocytometer; and the mononuclear cell count in the blood cell culture medium was adjusted. The blood cell culture medium containing the mononuclear cells was introduced into a syringe functioning as a variable-volume container, and the variable-volume container was aseptically connected to the container flow path through a resin tube functioning as a variable-volume container flow path. The volume of the variable-volume container was reduced in order to transfer, into the housing, the mononuclear cell-containing blood cell culture medium in the variable-volume container. After this, the container flow path was closed, the housing was placed in an incubator, and the mononuclear cells were two-dimensionally cultured in the housing from day 1 to day 7 at 37° C. On the 3rd day, blood cell culture medium was introduced into a variable-volume container and the variable-volume container was aseptically connected to the container flow path through a variable-volume container flow path. The volume of the variable-volume container was reduced in order to transfer, into the housing, the blood cell culture medium in the variable-volume container, thereby supplementing the blood cell culture medium in the housing.

A solution containing a Sendai virus (CytoTune-iPS 2.0 Sendai Reprogramming Kit, registered trademark, Thermo Fisher Scientific Inc.) capable of bringing about the expression of the OSKM initialization factors (OCT3/4, SOX2, KLF4, c-MYC) was introduced into a variable-volume container and the variable-volume container was aseptically connected to the container flow path through a variable-volume container flow path. The volume of the variable-volume container was reduced in order to transfer, into the housing, the Sendai virus-containing solution in the variable-volume container. The Sendai virus was added at an MOI of 5 to the mononuclear cells being cultured in the housing. This was followed by closure of the container flow path, and the housing was placed in an incubator and the cells were cultured at 34° C.

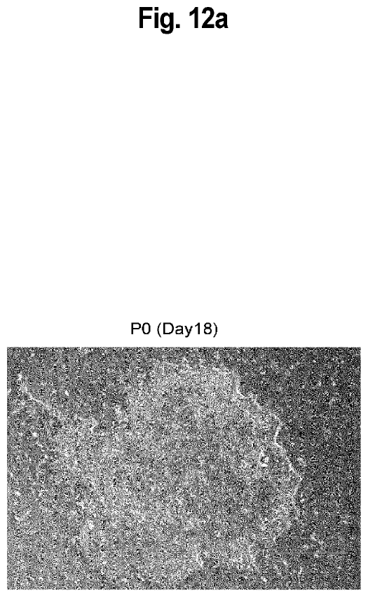

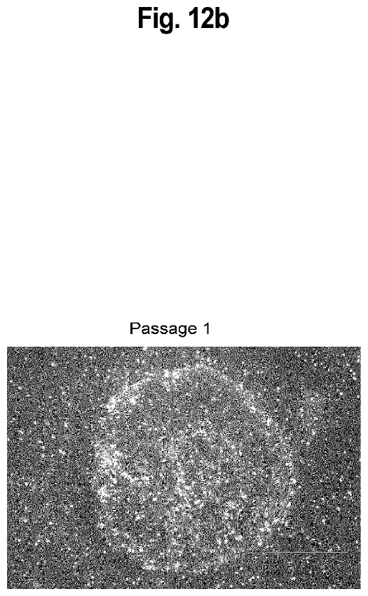

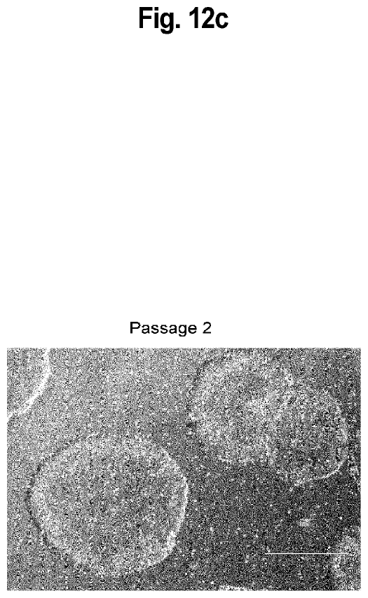

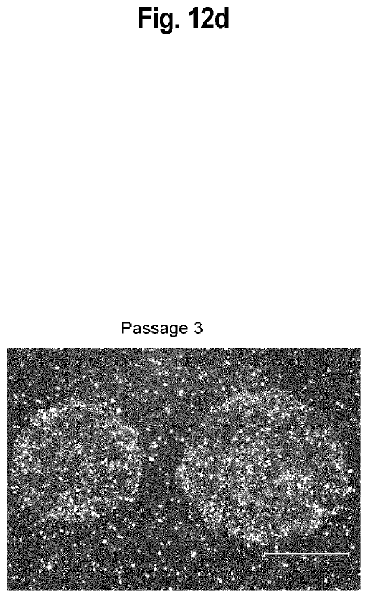

After two days after Sendai virus addition to the cells, an iPS cell culture medium was added into the housing. When culture medium exchange was carried out, an empty variable-volume container was aseptically connected to the housing and the culture medium in the housing was suctioned out into the variable-volume container. A culture medium-containing variable-volume container was then aseptically connected to the housing and the culture medium in the variable-volume container was supplied into the housing. iPS cell culture medium was added to the housing once every 2 days up to and including the 6th day. After this, the culture medium in the housing was exchanged every 2 days using an iPS cell culture medium. When culture medium exchange was carried out, an empty variable-volume container was aseptically connected to the housing and the culture medium in the housing was suctioned out into the variable-volume container. A culture medium-containing variable-volume container was then aseptically connected to the housing and the culture medium in the variable-volume container was supplied into the housing. From midway, the temperature of the incubator holding the housing was raised in steps to 37° C. and 38° C. After 8 days after infection, masses of stem cell-like cells had been produced. Almost all the cells had become TRA1-60-positive cells at the 18th day after infection and an iPS cell-like state was displayed. A cell photograph on the 14th day post-infection is given in FIG. 12(a).

After 14 days post-infection, a variable-volume container containing the cell detachment agent TrypLE Select was aseptically connected to the housing and the TrypLE Select was supplied into the housing. After allowing the housing to stand for 1 minute at room temperature, a solution containing the detached cells was suctioned out using a variable-volume container. The cells were then dispersed in an iPS cell culture medium, and the cell-containing iPS cell culture medium was recovered into a 15 mL tube. The cell count was measured using a hemocytometer; the concentration of the cell-containing solution was adjusted; and a first subculture was performed by seeding the cells into a housing at a concentration of not more than 0.25×10⁴ cells/cm². A cell photograph on the fifth day after the first subculture is shown in FIG. 12(b).

A second subculture was performed on the 5th day after the first subculture. FIG. 12(c) shows a cell photograph on the 5th day after the second subculture. A third subculture was performed on the 7th day after the second subculture. FIG. 12(d) shows a cell photograph on the fifth day after the third subculture.

### REFERENCE SIGNS LIST

10 Container
11 Housing
12 Lid
13 Filter
20 Container flow path
30, 31, 32, 33 Variable-volume container
40, 41, 42, 43 Variable-volume container flow path
50 Cutter
121, 132 Holder

1. A cell culture vessel comprising:
a container; and
a container flow path connected to the container for delivering a fluid into the container,
wherein at least a portion of the container is gas permeable.

2. The cell culture vessel according to claim 1, wherein the gas permeable portion of the container is impermeable to viruses.

3. The cell culture vessel according to claim 1 or 2, wherein the container comprises a housing having an opening and a lid for the opening.

4. The cell culture vessel according to claim 3, wherein the container flow path is connected to the housing of the container.

5. The cell culture vessel according to claim 3 or 4, wherein at least a portion of the housing is gas permeable.

6. The cell culture vessel according to claim 3, wherein the container flow path is connected to the lid for the container.

7. The cell culture vessel according to claim 3 or 6, wherein at least a portion of the lid is gas permeable.

8. The cell culture vessel according to any one of claims 1 to 7, wherein the container flow path is closable.

9. The cell culture vessel according to any one of claims 1 to 8, further comprising a variable-volume container connected to the container flow path.

10. The cell culture vessel according to claim 9, further comprising a variable-volume container flow path that is connected to the variable-volume container.

11. The cell culture vessel according to claim 10, wherein the variable-volume container flow path is closable.

12. The cell culture vessel according to claim 10 or 11, wherein the container flow path and the variable-volume container flow path are aseptically connected.

13. The cell culture vessel according to any one of claims 9 to 12, configured whereby the volume of the variable-volume container changes when a fluid in the variable-volume container is transferred into the container.

14. The cell culture vessel according to any one of claims 9 to 13, configured whereby the volume of the variable-volume container changes when a fluid in the container is transferred into the variable-volume container.

15. A cell culture method comprising:
preparing a cell culture vessel comprising a container and a container flow path connected to the container for delivering a fluid into the container, wherein at least a portion of the container is gas permeable;
connecting, to the container flow path, a variable-volume container that contains a cell in its interior;
transferring the cell into the container by contracting the variable-volume container; and
culturing the cell in the container.

16. The cell culture method according to claim 15, further comprising:
closing the container flow path after the cell has been transferred into the container.

17. The cell culture method according to claim 15 or 16, wherein a variable-volume container flow path is connected to the variable-volume container and the variable-volume container is connected to the container flow path by aseptically connecting the container flow path and the variable-volume container flow path.

18. The cell culture method according to claim 17, further comprising:
closing the variable-volume container flow path after the cell has been transferred into the container.

19. The cell culture method according to any one of claims 15 to 18, further comprising:
transferring culture medium in the container into the variable-volume container by expanding the variable-volume container.

20. The cell culture method according to any one of claims 15 to 19, wherein the container is placed in a carbon dioxide incubator during the culture of the cell in the container.

21. A method for initializing a cell comprising:
culturing a cell in the container of a cell culture vessel comprising a container and a container flow path connected to the container for delivering a fluid into the container, wherein at least a portion of the container is gas permeable;
connecting, to the container flow path, a variable-volume container that contains an initialization factor in its interior;
transferring the initialization factor into the container by contracting the variable-volume container; and
initializing the cell in the container.

22. The method for initializing a cell according to claim 21, further comprising:
closing the container flow path after the initialization factor has been transferred into the container.

23. The method for initializing a cell according to claim 21 or 22, wherein a variable-volume container flow path is connected to the variable-volume container and the variable-volume container is connected to the container flow path by aseptically connecting the container flow path and the variable-volume container flow path.

24. The method for initializing a cell according to claim 23, further comprising:
closing the variable-volume container flow path after the cell has been transferred into the container.

25. The method for initializing a cell according to any one of claims 21 to 24, wherein the container is placed in a carbon dioxide incubator for the initialization of the cell in the container.

26. A method for inducing cell differentiation, comprising:
culturing a cell in the container of a cell culture vessel comprising a container and a container flow path connected to the container for delivering a fluid into the container, wherein at least a portion of the container is gas permeable;
connecting, to the container flow path, a variable-volume container that contains a differentiation induction factor in its interior;
transferring the differentiation induction factor into the container by contracting the variable-volume container; and
inducing the differentiation of the cell in the container.

27. The method for inducing cell differentiation according to claim 26, further comprising:
closing the container flow path after the differentiation induction factor has been transferred into the container.

28. The method for inducing cell differentiation according to claim 26 or 27, wherein a variable-volume container flow path is connected to the variable-volume container and the variable-volume container is connected to the container flow path by aseptically connecting the container flow path and the variable-volume container flow path.

29. The method for inducing cell differentiation according to claim 28, further comprising:
closing the variable-volume container flow path after the cell has been transferred into the container.

30. The method for inducing cell differentiation according to any one of claims 26 to 29, wherein the container is placed in a carbon dioxide incubator during induction of the differentiation of the cell in the container.
